(19) United States
(12) Patent Application Publication
(10) Pub. No.: US 2025/0231202 A1
(43) Pub. Date: Jul. 17, 2025

(54) METHOD FOR PREDICTING THE POSTOPERATIVE RISK OF BRAIN-INJURED PATIENTS AND THE USE OF STEROIDS/PROTEINS FOR PREDICTING THE POSTOPERATIVE RISK OF BRAIN-INJURED PATIENTS

(71) Applicant: Taipei Medical University, Taipei City (TW)

(72) Inventor: Chien-Ying Chuang, Taipei City (TW)

(21) Appl. No.: 18/675,183

(22) Filed: May 28, 2024

(30) Foreign Application Priority Data
Jan. 17, 2024 (TW) 113101889

(51) Int. Cl. G01N 33/74; G01N 30/02; G01N 30/72; G01N 30/88; G01N 33/53; G01N 33/68

(52) U.S. Cl. CPC G01N 33/743; G01N 30/7233; G01N 33/53; G01N 33/6893; G01N 2030/027; G01N 2030/8813; G01N 2333/4712; G01N 2800/28; G01N 2800/50

(57) ABSTRACT

The present invention discloses a method for predicting the postoperative risk of brain-injured patients and a use of steroids/proteins for predicting the postoperative risk of brain-injured patients, mainly for testing the concentration of steroids/protein in patient samples. Acquire one-day in-vitro waste cerebrospinal fluid samples from brain-injured patients after surgery and conduct specific steroid and protein concentration tests. By confirming the concentration of specific steroid in the waste cerebrospinal fluid samples or the type and concentration of proteins detected therein, the postoperative risks of brain-injured patients can be predicted.

### FIELD OF THE INVENTION

The invention relates to a method and the use, particularly a method for predicting the postoperative risk of brain-injured patients and the use of steroids/proteins for predicting the postoperative risk of brain-injured patients.

### BACKGROUND OF THE INVENTION

Traumatic brain injury (TBI), also known as brain injury, refers to damage to the brain caused by external forces. Causes of traumatic brain injuries include slips and falls, traffic accidents, violent attacks, fights, physical exercises, etc., which may all cause traumatic brain injuries. Various emergencies caused by brain injuries can also cause traumatic brain injuries more severe. According to global statistics, traumatic brain injuries are the leading cause of death and disability in children and adolescents, with men suffering from traumatic brain injuries about twice as often as women. Although only half as many women suffer traumatic brain injuries as men, once a traumatic brain injury occurs, the symptoms are generally more severe.

Traumatic brain injury causes meningeal compression and blood vessel destruction, causing cell edema and necrosis. Nerve cells are damaged and ruptured, releasing pro-inflammatory cytokines, reactive oxygen species (ROS), ATP, and free nucleic acids. Within minutes, microglia cells under stimulation first move to the site to remove damaged cells, and gradually activate the immune response in the injured brain area.

Several hours after traumatic brain injury, the injured area gradually recruits neutrophils, releases ROS, matrix metalloproteinase (MMP), and pro-inflammatory cytokines, and destroying the blood-brain barrier. Moreover, these neutrophils entering the central nervous system will further exacerbate brain inflammation and immune response, inducing more neuronal cell death.

Days after traumatic brain injury, monocytes continue to recruit and release cellular inflammatory factors such as interleukin 1 (IL-1), IL-6, and tumor necrosis factor (TNF). In fact, during the entire brain injury process, these pro-inflammatory cytokines are not very helpful in repairing brain tissue after injury.

Not only that, but excessive immunity also interferes with the repair of damaged neurons. Even weeks after the injury, the concentration of pro-inflammatory cytokines may continue to remain at a high level, causing more extensive neuronal damage, causing secondary damage, and even turning into chronic inflammation. At the same time, excessive immune response can also promote the formation of astrocyte scar, which inhibits the regeneration of damaged nerve axons.

Methods to prevent traumatic brain injury include wearing seat belts when driving, wearing helmets when riding motorcycles, not drinking and driving, fall protection for the elderly, and safety protection measures for children.

Imaging technologies used to diagnose traumatic brain injury include X-ray computed tomography (CT) and magnetic resonance imaging (MRI). According to the aforementioned diagnostic methods, after the doctor confirms the brain injury, the available treatments include medication, emergency surgery, or surgery a few years later.

Among them, when patients with traumatic brain injury undergo emergency surgery, they usually need a dangerous observation period. During the dangerous observation period, they often become unconscious for unknown reasons, resulting in poor postoperative recovery and uncertain prognosis. The condition continues to worsen, eventually leading to the patient's death.

However, the above-mentioned patients had no abnormal conditions during the operation and no serious complications occurred when the operation was completed. Unfortunately, they continued to be comatose and eventually died, making the mortality rate of traumatic brain injury high.

Accordingly, how to predict the postoperative risks of traumatic brain injury patients after surgery based on the postoperative status of traumatic brain injury patients is a challenge faced by those skilled in the art.

### SUMMARY

An objective of the present invention is to provide a method for predicting the postoperative risk of brain-injured patients and a use of steroids/proteins for predicting the postoperative risk of brain-injured patients. Collect one-day in-vitro waste cerebrospinal fluid samples from brain-injured patients after surgery. According to the concentration of steroid in the waste cerebrospinal fluid samples, the postoperative risks in brain-injured patients can be predicted and hence assisting doctors to assess patents' postoperative conditions.

An objective of the present invention is to provide a method for predicting the postoperative risk of brain-injured patients and a use of steroids/proteins for predicting the postoperative risk of brain-injured patients. Collect one-day in-vitro waste cerebrospinal fluid samples from brain-injured patients after surgery. According to the types of proteins in the waste cerebrospinal fluid samples, the postoperative risks in brain-injured patients can be predicted and hence assisting doctors to assess patents' postoperative conditions.

To achieve the above objective, the present invention provides a method for predicting the postoperative risks of brain-injured patients, comprising steps of acquiring a clarified cerebrospinal fluid after centrifuging a waste cerebrospinal fluid sample; acquiring a steroid concentration by analyzing said clarified cerebrospinal fluid using a chromatography; and predicting a postoperative risk of a brain-injured patient according to said steroid concentration. Wherein said steroid concentration is a 21-deoxycortisol concentration; and when said 21-deoxycortisol concentration is greater than 0.5 ng/mL, said brain-injured patient is predicted to have a high risk level.

According to an embodiment of the present invention, in the step of acquiring a steroid concentration by analyzing said clarified cerebrospinal fluid using a chromatography, said chromatography adopts a liquid chromatography/tandem mass spectrometer (LC-MS/MS).

To achieve the above objective, the present invention provides A method for predicting the postoperative risks of brain-injured patients, comprising steps of acquiring a clarified cerebrospinal fluid after centrifuging a waste cerebrospinal fluid sample; acquiring a steroid concentration by analyzing said clarified cerebrospinal fluid using an enzyme-linked immunosorbent assay; and predicting a postoperative risk of a brain-injured patient according to said steroid concentration. Wherein said steroid concentration is a dehydroepiandrosterone concentration; and when said dehydroepiandrosterone concentration is less than 50 ng/mL, said brain-injured patient is predicted to have a high risk level.

According to an embodiment of the present invention, in the step of acquiring a steroid concentration by analyzing said clarified cerebrospinal fluid using an enzyme-linked immunosorbent assay, said enzyme-linked immunosorbent assay adopts a DHEA sulfate (DHEA-S) detection reagent.

To achieve the above objective, the present invention provides a method for predicting the postoperative risks of brain-injured patients, comprising steps of acquiring a clarified cerebrospinal fluid after centrifuging a waste cerebrospinal fluid sample; acquiring a test sample by performing a protein purification procedure on the clarified cerebrospinal fluid; and analyzing the types of proteins in the test sample using a protein immunoassay; and predicting a brain-injured patient to have a high risk level when said test sample contains a protein. Wherein said protein is selected from the group consisting of an acetylated tubulin α, a myosin heavy chain 10, and a zinc finger protein 179.

According to an embodiment of the present invention, the step of analyzing the types of proteins in the test sample using a protein immunoassay comprises a step of identifying said acetylated tubulin α in said test sample using a monoclonal antibody of acetylated tubulin α.

According to an embodiment of the present invention, the step of analyzing the types of proteins in the test sample using a protein immunoassay comprises a step of identifying said myosin heavy chain 10 in said test sample using a polyclonal antibody of myosin heavy chain 10.

According to an embodiment of the present invention, the step of analyzing the types of proteins in the test sample using a protein immunoassay comprises a step of identifying said zinc finger protein 179 in said test sample using an antibody of zinc finger protein 179.

To achieve the above objective, the present invention provides a use of steroids for predicting the postoperative risks of brain-injured patients. When a 21-deoxycortisol concentration in a waste cerebrospinal fluid sample is greater than 0.5 ng/mL, a brain-injured patient is predicted to have a high risk level. The steroid is 21-deoxycortisol.

To achieve the above objective, the present invention provides a use of steroids for predicting the postoperative risks of brain-injured patients. When a dehydroepiandrosterone concentration in a waste cerebrospinal fluid sample is less than 50 ng/mL, a brain-injured patient is predicted to have a high risk level. The steroid is dehydroepiandrosterone.

To achieve the above objective, the present invention provides a use of proteins for predicting the postoperative risks of brain-injured patients. When a waste cerebrospinal fluid sample contains a protein, a brain-injured patient is predicted to have a high risk level. The protein is acetylated tubulin α.

To achieve the above objective, the present invention provides a use of proteins for predicting the postoperative risks of brain-injured patients. When a waste cerebrospinal fluid sample contains a protein, a brain-injured patient is predicted to have a high risk level. The protein is myosin heavy chain 10.

To achieve the above objective, the present invention provides a use of proteins for predicting the postoperative risks of brain-injured patients. When a waste cerebrospinal fluid sample contains a protein, a brain-injured patient is predicted to have a high risk level. The protein is zinc finger protein 179.

### BRIEF DESCRIPTION OF THE FIGURES

FIG. 1A shows a flowchart of the method according to a first embodiment of the present invention;

FIG. 1B shows a schematic diagram of the experimental results (21-deoxycortisol concentration) according a first embodiment of the present invention;

FIG. 2A shows a flowchart of the method according to a second embodiment of the present invention;

FIG. 2B shows a schematic diagram of the experimental results (dehydroepiandrosterone sulfate (DHEA-S) concentration) according a second embodiment of the present invention;

FIG. 3A shows a flowchart of the method according to a third embodiment of the present invention; and

FIG. 3B shows a schematic diagram of the experimental results according a third embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

After patients with traumatic brain injury undergo emergency surgery and during the dangerous observation period, they often become unconscious for unknown reasons, resulting in poor postoperative recovery and uncertain prognosis. The condition continues to worsen, eventually leading to the patient's death.

The advantage of the present invention is that by collect and analyzing one-day in-vitro waste cerebrospinal fluid samples from brain-injured patients after surgery, according to the concentration of steroid or the types of proteins in the waste cerebrospinal fluid samples, the postoperative risks in brain-injured patients can be predicted and hence assisting doctors to assess patents' postoperative conditions.

In the following description, various embodiments of the present invention are described using figures for describing the present invention in detail. Nonetheless, the concepts of the present invention can be embodied by various forms. Those embodiments are not used to limit the scope and range of the present invention.

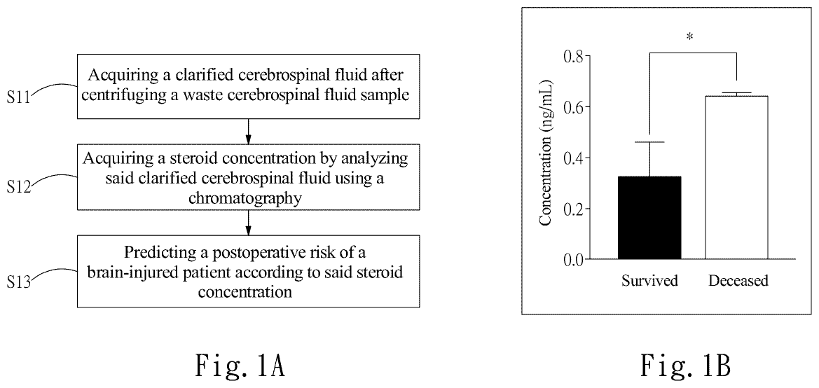

First, please refer to FIG. 1A, which shows a flowchart of the method according to a first embodiment of the present invention. As shown in FIG. 1A, the first embodiment comprises steps of:
Step S11: Acquiring a clarified cerebrospinal fluid after centrifuging a waste cerebrospinal fluid sample;
Step S12: Acquiring a steroid concentration by analyzing said clarified cerebrospinal fluid using a chromatography; and
Step S13: Predicting a postoperative risk of a brain-injured patient according to said steroid concentration.

In the step S11, acquire a clarified cerebrospinal fluid after centrifuging a waste cerebrospinal fluid sample. said waste cerebrospinal fluid sample is stored in the testing refrigerator and comes from the waste cerebrospinal fluid isolated from a brain-injured patient within one day after surgery. Said waste cerebrospinal fluid sample is centrifuged under a centrifugal condition to remove the red blood cells therein. Then take the upper layer of said clarified cerebrospinal fluid (approximately 200 μl). Said centrifugation condition is centrifugation at 400 G for ten minutes at 4° C.

Next, according to the steps S12 and S13 of the first embodiment, acquire a steroid concentration by analyzing said clarified cerebrospinal fluid using a chromatography and predicting a postoperative risk in said brain-injured patient according to said steroid concentration. Then said postoperative risk level can assist doctors to assess patents' postoperative conditions.

According to the first embodiment, said chromatography adopts a liquid chromatograph/tandem mass spectrometer (LC-MS/MS). A set of analytical instruments composed of a liquid chromatograph and a mass spectrometer. The principle is to use a high pressure pump to pressurize to generate power to make the liquid mobile phase flow. In this process, the mobile phase will drive the analyte to flow through the stationary phase in the column. Since the forces between different analytes and the stationary phase will be different, there will be different retention times in the column to achieve the purpose of separation. Then a mass spectrometer is used to detect. It can be applied to the qualitative and quantitative analysis of trace substances. Therefore, according to the first embodiment, said chromatograph/tandem mass spectrometer is used to acquire said steroid concentration in said clarified cerebrospinal fluid.

Said steroid concentration is a 21-deoxycortisol concentration. The first embodiment discloses a use of said steroids to predict said postoperative risks in said brain-injured patients. According to the first embodiment, when said 21-deoxycortisol concentration is greater than 0.5 ng/mL, said brain-injured patient is predicted to have a high risk level, indicating increased likelihood of falling into coma and death.

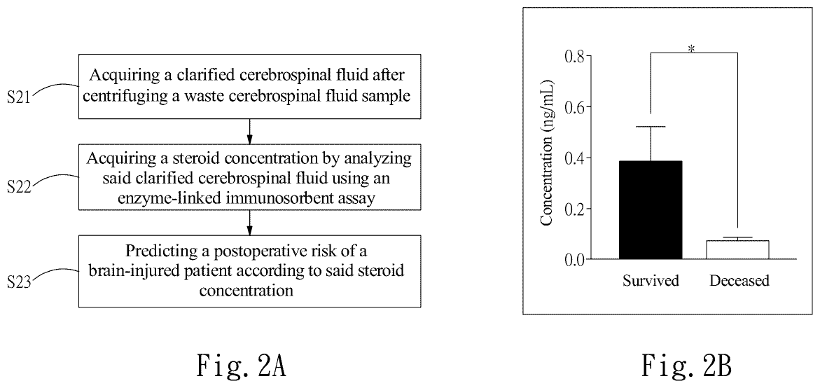

Next, please refer to FIG. 2A, which shows a flowchart of the method according to a second embodiment of the present invention. As shown in FIG. 2A, the second embodiment comprises steps of:
Step S21: Acquiring a clarified cerebrospinal fluid after centrifuging a waste cerebrospinal fluid sample;
Step S22: Acquiring a steroid concentration by analyzing said clarified cerebrospinal fluid using an enzyme-linked immunosorbent assay; and
Step S23: Predicting a postoperative risk of a brain-injured patient according to said steroid concentration.

The step S21 according to the second embodiment is identical to the first embodiment. Hence, the details will not be repeated.

Next, according to the steps S22 and S23 of the second embodiment, acquire said steroid concentration by analyzing said clarified cerebrospinal fluid using an enzyme-linked immunosorbent assay and predicting a postoperative risk in said brain-injured patient according to said steroid concentration. Then said postoperative risk level can assist doctors to assess patents' postoperative conditions.

According to the second embodiment, said enzyme-linked immunosorbent assay adopts a DHEA sulfate (DHEA-S) detection reagent. Said enzyme-linked immunosorbent assay (ELISA) is also called the enzyme immunoassay. It is an analytical method that utilizes the specific bonding characteristics between antigens and antibodies to detect specimens. Since the antigen or antibody bound to a solid carrier (usually a plastic well plate) can still have immune activity, the binding mechanism is designed. Combined with an enzyme color reaction, the presence or absence of specific antigens or antibodies can be displayed, and the depth of color can be used for quantitative analysis. Thereby, according to the second embodiment, said steroid concentration in said clarified cerebrospinal fluid can be acquired by using said enzyme-linked immunosorbent assay.

Said steroid concentration according to the second embodiment is a dehydroepiandrosterone (DHEA) concentration. The second embodiment discloses a use of said steroids to predict said postoperative risks in said brain-injured patients. According to the second embodiment, when said dehydroepiandrosterone concentration is less than 50 ng/mL, said brain-injured patient is predicted to have a high risk level, indicating increased likelihood of falling into coma and death.

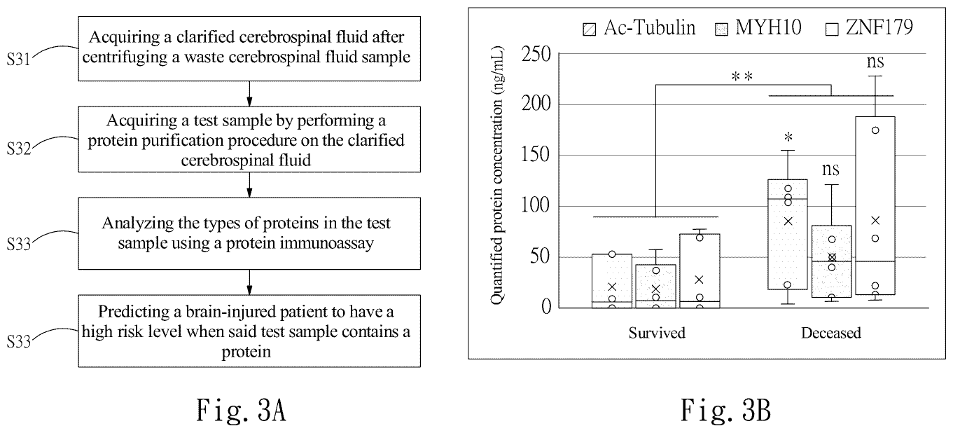

Next, please refer to FIG. 3A, which shows a flowchart of the method according to a third embodiment of the present invention. As shown in FIG. 3A, the third embodiment comprises steps of:
Step S31: Acquiring a clarified cerebrospinal fluid after centrifuging a waste cerebrospinal fluid sample;
Step S32: Acquiring a test sample by performing a protein purification procedure on the clarified cerebrospinal fluid;
Step S33: Analyzing the types of proteins in the test sample using a protein immunoassay; and
Step S34: Predicting a brain-injured patient to have a high risk level when said test sample contains a protein.

The step S31 according to the third embodiment is identical to the first embodiment. Hence, the details will not be repeated.

Next, according to the steps S32 and S33 of the third embodiment, acquire a test sample by performing a protein purification procedure on said clarified cerebrospinal fluid. Then analyze the types of proteins in said test sample using a protein immunoassay. When said test sample contains a protein, predict said brain-injured patient to have a high risk level. By analyzing and detecting the types of proteins in said test sample, the risk level of said postoperative risk of said brain-injured patient can be predicted. Then the risk level can assist doctors to assess patents' postoperative conditions.

Said protein is selected from the group consisting of an acetylated tubulin α (acetyl-α-Tubulin (Lys40)), a myosin heavy chain 10 (MYH10), and a zinc finger protein 179 (ZnF179, also known as RNF112 or neurolastin).

In addition, according to the third embodiment, said protein purification procedure is a series of processes that separate one or several proteins from a complex mixture (usually cells, tissues, or whole organs). Said protein purification procedure is capable of separating proteins and non-proteins in said clarified cerebrospinal fluid. part, and finally separate the required protein from all other proteins. According to the prior art, depending on the fragility of protein and the stability of cell, one of the following methods can be selected: salting out (hydrophobic interaction), colloid filtration, and affinity chromatography are used for protein purification.

Furthermore, according to the third embodiment, the protein immunoassay uses a specific antibody to mark a specific protein through the ELISA analysis and then uses the marker to confirm the types of the proteins. Therefore, according to the present embodiment, the protein immunoassay is used to analyze and acquire the types of proteins in the clarified cerebrospinal fluid.

Next, to identify whether said test sample contains said acetylated tubulin α. Said protein immunoassay can use an acetylated tubulin α monoclonal antibody (6-11B-1). To identify whether said test sample contains said myosin heavy chain 10, said protein immunoassay can use a myosin heavy chain 10 multi-strain antibody (19673-1-AP). In addition, to identify whether said test sample contains a ring finger protein 112, said protein immunoassay can use a zinc finger protein 179 (aa1-30) antibody (LS-C324716).

The third embodiment discloses a use of proteins for predicting the postoperative risks of brain-injured patients. A high risk level indicates increased likelihood of falling into coma and death. Said protein is selected from the group consisting of said acetylated tubulin α, said myosin heavy chain 10, and said zinc finger protein 179.

After patients with traumatic brain injury undergo emergency surgery and during the dangerous observation period, they often become unconscious for unknown reasons, resulting in poor postoperative recovery and uncertain prognosis. The condition continues to worsen, eventually leading to the patient's death.

The advantage of the first, second, and third embodiments is that by collect and analyzing one-day in-vitro waste cerebrospinal fluid samples from brain-injured patients after surgery, according to the concentration of steroid or the types of proteins in the waste cerebrospinal fluid samples, the postoperative risks in brain-injured patients can be predicted and hence assisting doctors to assess patents' postoperative conditions and increasing the mortality rate of brain-injured patients.

In the following, some embodiments of the present invention will be described.

A number of brain-injured patients (12, all of whom were judged using the Glasgow Coma Scale (GCS) on admission, with scores between 6 and 7), obtained the results within one day of the completion of the operation. After centrifuging said waste cerebrospinal fluid sample isolated from the patient with brain injury, said clarified cerebrospinal fluid is taken out for analysis and detection.

The embodiments are described as follows.

### Embodiment 1

### Analyze Said Waste Cerebrospinal Fluid Sample Using Said Liquid Chromatograph/Tandem Mass Spectrometer (LC-MS/MS)

FIG. 1B shows a schematic diagram of the experimental results according a first embodiment of the present invention. Table 1 shows the 21-deoxycortisol concentration in said clarified cerebrospinal fluid of said brain-injured patients.

TABLE 1
21-deoxycortisol concentration and postoperative
condition in clarified cerebrospinal fluid

| | Patient No. | | | | | |
|---|---|---|---|---|---|---|
| | #1 | #2 | #3 | #4 | #5 | #6 |
| 21- | 0.39 | 0.22 | 0.42 | 0.65 | 0.65 | 0.62 |
| deoxycortisol | | | | | | |
| Concentration | | | | | | |
| (ng/mL) | | | | | | |
| Postoperative | Survived | Survived | Survived | Deceased | Deceased | Deceased |
| Condition | | | | | | |

From the experimental results presented in the above Table 1 and FIG. 1B (t-test:**P<0.01), it is understood that when said 21-deoxycortisol concentration is higher than 0.5 ng/mL, the patient's postoperative condition is poor and the mortality rate is high.

### Embodiment 2

### Analyze Said Cerebrospinal Fluid Sample Using Said Enzyme-Linked Immunosorbent Assay

FIG. 2B shows a schematic diagram of the experimental results according a second embodiment of the present invention. Table 2 shows the DHEA concentration in said clarified cerebrospinal fluid of said brain-injured patients.

TABLE 2
DHEA concentration and postoperative condition in clarified cerebrospinal fluid

| | Patient No. | | | | | |
|---|---|---|---|---|---|---|
| | #1 | #2 | #3 | #4 | #5 | #6 |
| DHEA | 66.55 | 94.51 | 56.24 | 10.51 | 9.94 | 16.07 |
| Concentration | | | | | | |
| (ng/mL) | | | | | | |
| Postoperative | Survived | Survived | Survived | Deceased | Deceased | Deceased |
| Condition | | | | | | |

From the experimental results presented in the above Table 2 and FIG. 2B (t-test:**P<0.01), it can be understood that when said dehydroepiandrosterone concentration is less than 50 ng/mL, the patient's postoperative condition is poor and the mortality rate is high.

### Embodiment 3

### Acquire Said Test Sample by Performing Said Protein Purification Procedure on Said Clarified Cerebrospinal Fluid and Analyze the Types of Proteins in Said Test Sample Using Said Protein Immunoassay.

FIG. 3B shows a schematic diagram of the experimental results according a third embodiment of the present invention. From the experimental results presented in FIG. 3B (t-test: *p<0.05, **p<0.01), it can be understood that when said test sample contains a large amount of the proteins selected from the group consisting of said acetylated tubulin α (Ac-Tubulin), said myosin heavy chain 10(MYH10), and said zinc finger protein 179(ZNF179), the patient's postoperative condition is poor and the mortality rate is high. Said acetylated tubulin α, said myosin heavy chain 10 and said zinc finger protein 179 according to the present invention are all expressed in cells and should not be detected in cerebrospinal fluid samples under normal conditions. However, when brain cells are damaged, intracellular substances will have the opportunity to be measured in cerebrospinal fluid samples, and the patient's postoperative condition will be represented based on the amount of measured expression.

In the past, during the hospitalization of patients with brain injury, some often continued to deteriorate due to unknown reasons and eventually died. Therefore, precise molecular markers were searched for brain-injured patients to quickly predict the post-injury risk level, which helps doctors classify the patient's brain injury accurately and allocate medical resources appropriately, thereby reducing the mortality rate of patients with brain injury.

The present invention provides a method for predicting the postoperative risks of brain-injured patients and a use of steroids/proteins for predicting the postoperative risks of brain-injured patients. Acquire one-day in-vitro waste cerebrospinal fluid samples from brain-injured patients after surgery. According to the steroid concentration (21-deoxycortisol, dehydroepiandrosterone) or the types of proteins (acetylated tubulin α, zinc finger protein 179, myosin heavy chain 10) in the cerebrospinal fluid samples, the postoperative risks in brain-injured patients can be predicted and hence assisting doctors to assess patents' postoperative conditions.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

1. A method for predicting the postoperative risks of brain-injured patients, comprising steps of:
acquiring a clarified cerebrospinal fluid after centrifuging a waste cerebrospinal fluid sample;
acquiring a steroid concentration by analyzing said clarified cerebrospinal fluid using a chromatography; and
predicting a postoperative risk of a brain-injured patient according to said steroid concentration;
wherein said steroid concentration is a 21-deoxycortisol concentration; and when said 21-deoxycortisol concentration is greater than 0.5 ng/mL, said brain-injured patient is predicted to have a high risk level.

2. The method for predicting the postoperative risks of brain-injured patients of claim 1, wherein said step of acquiring a steroid concentration by analyzing said clarified cerebrospinal fluid using a chromatography, said chromatography adopts a liquid chromatography/tandem mass spectrometer (LC-MS/MS).

3. A method for predicting the postoperative risks of brain-injured patients, comprising steps of:
acquiring a clarified cerebrospinal fluid after centrifuging a waste cerebrospinal fluid sample;
acquiring a steroid concentration by analyzing said clarified cerebrospinal fluid using an enzyme-linked immunosorbent assay; and
predicting a postoperative risk of a brain-injured patient according to said steroid concentration;
wherein said steroid concentration is a dehydroepiandrosterone concentration; and
when said dehydroepiandrosterone concentration is less than 50 ng/mL, said brain-injured patient is predicted to have a high risk level.

4. The method for predicting the postoperative risks of brain-injured patients of claim 3, where in said step of acquiring a steroid concentration by analyzing said clarified cerebrospinal fluid using an enzyme-linked immunosorbent assay, said enzyme-linked immunosorbent assay adopts a DHEA sulfate (DHEA-S) detection reagent.

5. A method for predicting the postoperative risks of brain-injured patients, comprising steps of:
acquiring a clarified cerebrospinal fluid after centrifuging a waste cerebrospinal fluid sample;
acquiring a test sample by performing a protein purification procedure on the clarified cerebrospinal fluid; and
analyzing the types of proteins in the test sample using a protein immunoassay; and
predicting a brain-injured patient to have a high risk level when said test sample contains a protein;
wherein said protein is selected from the group consisting of an acetylated tubulin α, a myosin heavy chain 10, and a zinc finger protein 179.

6. The method for predicting the postoperative risks of brain-injured patients of claim 5, wherein said step of analyzing the types of proteins in the test sample using a protein immunoassay comprises a step of identifying said acetylated tubulin α in said test sample using a monoclonal antibody of acetylated tubulin α.

7. The method for predicting the postoperative risks of brain-injured patients of claim 5, wherein said step of analyzing the types of proteins in the test sample using a protein immunoassay comprises a step of identifying said myosin heavy chain 10 in said test sample using a polyclonal antibody of myosin heavy chain 10.

8. The method for predicting the postoperative risks of brain-injured patients of claim 5, wherein said step of analyzing the types of proteins in the test sample using a protein immunoassay comprises a step of identifying said zinc finger protein 179 in said test sample using an antibody of zinc finger protein 179.

9. A use of steroids for predicting the postoperative risks of brain-injured patients, wherein when a 21-deoxycortisol concentration in a waste cerebrospinal fluid sample is greater than 0.5 ng/mL, a brain-injured patient is predicted to have a high risk level; and said steroid is a 21-deoxycortisol.

10. A use of steroids for predicting the postoperative risks of brain-injured patients, wherein when a dehydroepiandrosterone concentration in a waste cerebrospinal fluid sample is less than 50 ng/mL, a brain-injured patient is predicted to have a high risk level; and said steroid is a dehydroepiandrosterone.

11. A use of proteins for predicting the postoperative risks of brain-injured patients, wherein when a waste cerebrospinal fluid sample contains a protein, a brain-injured patient is predicted to have a high risk level; and said protein is an acetylated tubulin α.

12. A use of proteins for predicting the postoperative risks of brain-injured patients, wherein when a waste cerebrospinal fluid sample contains a protein, a brain-injured patient is predicted to have a high risk level; and said protein is a myosin heavy chain 10.

13. A use of proteins for predicting the postoperative risks of brain-injured patients, wherein when a waste cerebrospinal fluid sample contains a protein, a brain-injured patient is predicted to have a high risk level; and said protein is a zinc finger protein 179.
